(19) United States
(12) Patent Application Publication
(10) Pub. No.: US 2021/0251684 A1
(43) Pub. Date: Aug. 19, 2021

(54) ARTICULATING SURGICAL INSTRUMENT AND METHOD OF ASSEMBLING THE SAME

(71) Applicant: Covidien LP, Mansfield, MA (US)

(72) Inventor: Daniel A. Joseph, Golden, CO (US)

(21) Appl. No.: 17/129,398

(22) Filed: Dec. 21, 2020

Related U.S. Application Data
(60) Provisional application No. 62/978,569, filed on Feb. 19, 2020.

(51) Int. Cl. A61B 18/14

(52) U.S. Cl. CPC A61B 18/1445; A61B 2017/00526

(57) ABSTRACT

An articulating surgical instrument includes a distal retainer, a proximal shaft segment, a first articulating section extending between the distal retainer and the proximal shaft segment, a proximal base extending proximally from the proximal shaft segment, a proximal retainer, a second articulating section extending between the proximal base and proximal retainer. A plurality of articulation cables is secured at distal end portions thereof to the distal retainer and at proximal end portions thereof to the proximal retainer. The proximal shaft segment, first articulating section, proximal base, and second articulating section cooperate to define a central longitudinal lumen therethrough and a plurality of radial longitudinal lumens therethrough that are radially disposed about the central longitudinal lumen. The articulation cables extend through the radial longitudinal lumens. Articulation of the second articulating section affects articulation of the first articulating section.

### CROSS-REFERENCE TO RELATED APPLICATIONS

This application claims the benefit of, and priority to, U.S. Provisional Patent Application No. 62/978,569, filed on Feb. 19, 2020, the entire contents of which are hereby incorporated herein by reference.

### FIELD

The present disclosure is generally related to surgical instruments and, more particularly, to articulating shaft-based surgical instruments and methods of assembling the same.

### BACKGROUND

Shaft-based surgical instruments, including robotic shaft-based surgical instruments, have become widely used by surgeons in endoscopic and other surgical procedures because they enable surgery to be less invasive. As a direct result thereof, the surgery minimizes trauma to the patient and reduces patient recovery time and hospital costs.

Some shaft-based surgical instruments incorporate rotation and/or articulation features, thus enabling rotation and/or articulation of an end effector assembly of the surgical instrument relative to a proximal portion the surgical instrument to enable better positioning of the end effector assembly for performing a surgical task within a surgical site.

### SUMMARY

The present disclosure generally relates to articulating shaft-based surgical instruments and methods of assembling the same. As used herein, the term “distal” refers to the portion that is described which is farther from an operator (whether a human surgeon or a surgical robot), while the term “proximal” refers to the portion that is being described which is closer to the operator. Terms including “generally,” “about,” “substantially,” and the like, as utilized herein, are meant to encompass variations, e.g., manufacturing tolerances, material tolerances, use and environmental tolerances, measurement variations, and/or other variations, up to and including plus or minus 10 percent. Further, any or all of the aspects described herein, to the extent consistent, may be used in conjunction with any or all of the other aspects described herein.

Provided in accordance with aspects of the present disclosure is an articulating surgical instrument including a distal retainer, a proximal shaft segment, a first articulating section extending between the distal retainer and the proximal shaft segment, a proximal base extending proximally from the proximal shaft segment, a proximal retainer, a second articulating section extending between the proximal base and proximal retainer. A plurality of articulation cables is secured at distal end portions thereof to the distal retainer and at proximal end portions thereof to the proximal retainer. The proximal shaft segment, the first articulating section, the proximal base, and the second articulating section cooperate to define a central longitudinal lumen therethrough and a plurality of radial longitudinal lumens therethrough that are radially disposed about the central longitudinal lumen. Each articulation cable extends through one of the radial longitudinal lumens. Articulation of the second articulating section affects articulation of the first articulating section.

In an aspect of the present disclosure, the proximal base and/or the proximal shaft segment defines a solid body except for the portion of the central longitudinal lumen and portions of the radial longitudinal lumens extending therethrough.

In another aspect of the present disclosure, the proximal base tapers in diameter from a maximum diameter to a minimum diameter in a proximal-to-distal direction. In such aspects, the first articulating section may define the minimum diameter and the second articulating section may define the maximum diameter such that articulation of the second articulating section affects amplified articulation of the first articulating section.

In yet another aspect of the present disclosure, the radial longitudinal lumens are open channels along at least portions of lengths thereof.

In still another aspect of the present disclosure, the distal retainer, the proximal shaft segment, the first and second articulating sections, the proximal base, and the proximal retainer cooperate to define an overall length. In such aspects, a constant outer diameter of less than about 3.5 mm may be defined along at least 30% of the overall length.

In still yet another aspect of the present disclosure, the first articulating section defines a bend radius of at least 4.5 mm. Additionally or alternatively, the first articulating section defines a bend ratio of less than about 2.0.

In another aspect of the present disclosure, at least one actuation component extends through the central longitudinal lumen. In aspects, at least two actuation components extend through the central longitudinal lumen.

In still another aspect of the present disclosure, the first and/or second articulating section includes a plurality of articulation links configured to articulate omnidirectionally relative to one another.

In yet another aspect of the present disclosure, each articulation link of the plurality of articulation links defines a semi-spherical recess at one end and semi-spherical protrusion at the other end.

A method of assembling an articulating surgical instrument provided in accordance with the present disclosure includes securing a plurality of articulating cables within a distal retainer, routing the plurality of articulation cables through at least one shaft segment and at least one articulating section, routing the plurality of articulation cables through a proximal retainer such that proximal end portions of the plurality of articulation cables extend through and proximally from the proximal retainer (wherein each articulation cable of the plurality of articulation cables is routed separately through the proximal retainer), applying tension at the proximal end portions of the plurality of articulation cables to thereby uniformly tension each articulation cable of the plurality of articulation cables, and securing the proximal end portion of each articulation cable to the proximal retainer to maintain the uniform tension on each articulation cable.

In an aspect of the present disclosure, after securing the proximal end portion of each articulation cable, the method further includes cutting off any excess cable at the proximal end portion of each cable.

In another aspect of the present disclosure, routing the plurality of articulation cables through the at least one shaft segment and at least one articulating section includes routing each articulation cable through a separate lumen.

In still another aspect of the present disclosure, the method further includes routing the plurality of articulation cables through a proximal washer before the proximal retainer. In such aspects, the proximal washer may act as a thermal barrier to distal components during the securing of the proximal end portions of each articulation cable to the proximal retainer.

In yet another aspect of the present disclosure, securing of the proximal end portions of each articulation cable to the proximal retainer includes at least one of: crimping, bonding, brazing, soldering, welding, or set screw engagement.

In another aspect of the present disclosure, applying tension includes individually tensioning each of the articulation cables. Further, individually tensioning each of the articulation cables may include individually adjusting a tension on at least one of the articulation cables.

The details of one or more aspects of the disclosure are set forth in the accompanying drawings and the description below. Other features, objects, and advantages of the techniques described in this disclosure will be apparent from the description and drawings, and from the claims.

### BRIEF DESCRIPTION OF DRAWINGS

The above and other aspects and features of the present disclosure will become more apparent in view of the following detailed description when taken in conjunction with the accompanying drawings wherein like reference numerals identify similar or identical elements.

FIG. 1A is a perspective view of a shaft-based articulating surgical instrument provided in accordance with the present disclosure wherein an end effector assembly thereof is disposed in an un-articulated position;

FIG. 1B is a perspective view of the shaft-based articulating surgical instrument of FIG. 1A wherein the end effector assembly is disposed in an articulated position;

FIG. 2 is a schematic illustration of a robotic surgical system provided in accordance with the present disclosure;

FIG. 3 is a perspective, longitudinal, cross-sectional view of a shaft assembly configured for use with the shaft-based articulating surgical instrument of FIG. 1A, the robotic surgical system of FIG. 2, or any other suitable shaft-based surgical instrument;

FIG. 4A is a side view of the shaft assembly of FIG. 3, disposed in an un-articulated position;

FIG. 4B is a side, longitudinal, cross-sectional view of the shaft assembly of FIG. 3, disposed in the un-articulated position;

FIG. 5A is a side view of the shaft assembly of FIG. 3, disposed in an articulated position, with the articulation cables removed;

FIG. 5B is a side, longitudinal, cross-sectional view of the shaft assembly of FIG. 3, disposed in the articulated position, with the articulation cables removed;

FIG. 6 is a perspective view of the shaft assembly of FIG. 3 with portions removed;

FIG. 7 is another perspective view of the shaft assembly of FIG. 3 with portions removed;

FIG. 8 is a perspective view illustrating a portion of the shaft assembly of FIG. 3 during assembly thereof; and

FIG. 9 is a flow diagram illustrating a method of assembly in accordance with the present disclosure.

### DETAILED DESCRIPTION

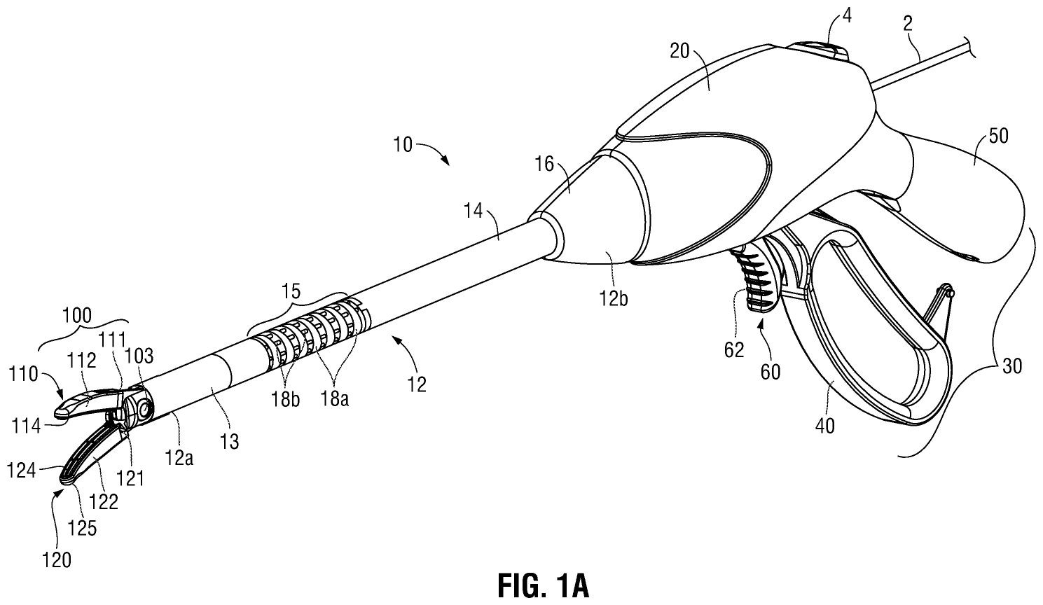

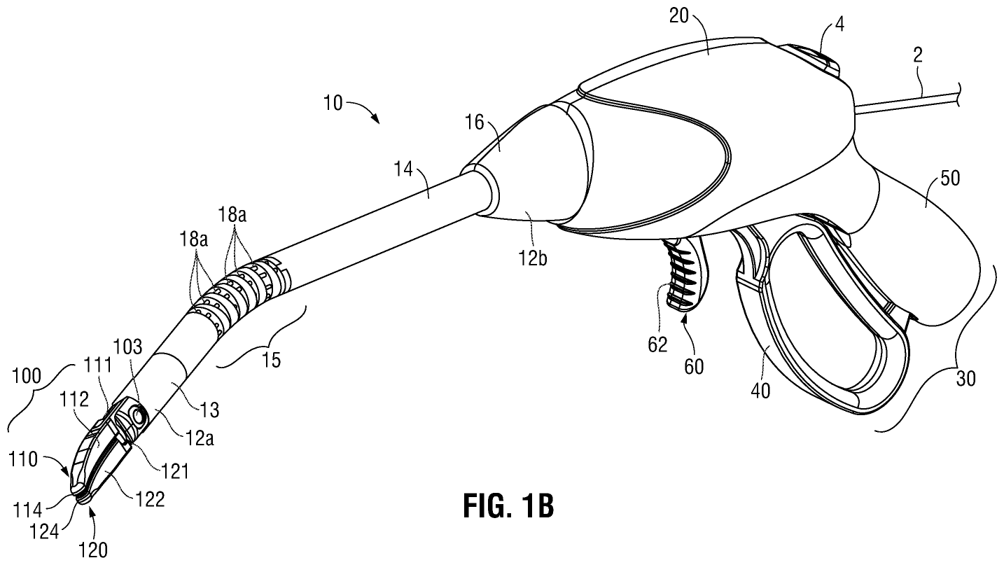

Referring generally to FIGS. 1A and 1B, a shaft-based surgical instrument exemplifying the aspects and features of the present disclosure is shown generally identified by reference numeral 10. For the purposes herein, surgical instrument 10 is generally described. Aspects and features of surgical instrument 10 not germane to the understanding of the present disclosure are omitted to avoid obscuring the aspects and features of the present disclosure in unnecessary detail.

Surgical instrument 10 includes a housing 20, a handle assembly 30, a trigger assembly 60, an activation switch 4, and an end effector assembly 100. Surgical instrument 10 further includes a shaft assembly 12 having a distal end portion 12a configured to mechanically engage end effector assembly 100 and a proximal end portion 12b that engages housing 20. Surgical instrument 10 also includes cable 2 that connects surgical instrument 10 to an energy source (not shown), e.g., a generator or other suitable power source, although surgical instrument 10 may alternatively be configured as a battery-powered device. Cable 2 includes a wire (or wires) (not shown) extending therethrough that has sufficient length to extend through shaft assembly 12 in order to provide energy to one or both tissue-treating plates 114, 124 of jaw members 110, 120, respectively, of end effector assembly 100. Activation switch 4 is coupled to tissue-treating plates 114, 124 and the source of energy for selectively activating the supply of energy to jaw members 110, 120 for treating, e.g., cauterizing, coagulating/desiccating, and/or sealing, tissue.

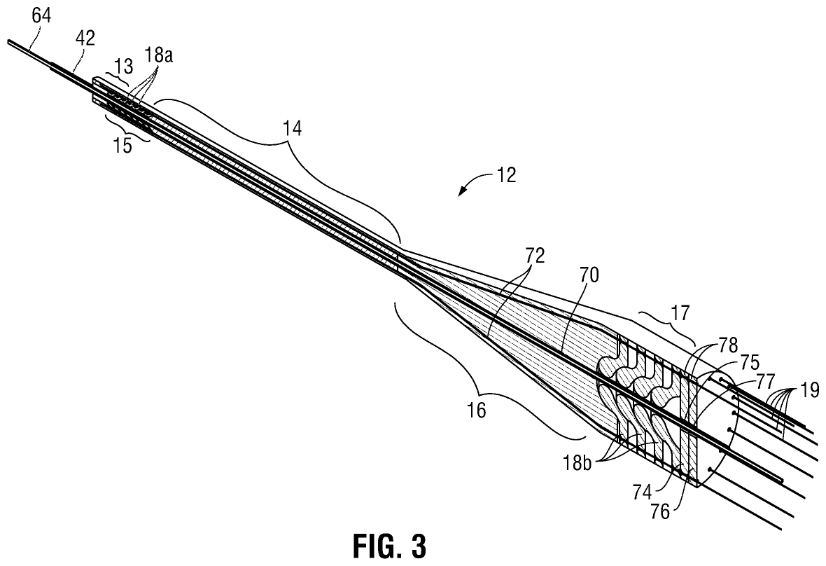

Shaft assembly 12 of surgical instrument 10 includes a distal segment 13 positioned towards distal end portion 12a thereof, a proximal segment 14 positioned towards proximal end portion 12b thereof, a distal articulating section 15 disposed between the distal and proximal segments 13, 14, respectively, a proximal base 16, and a proximal articulating section 17 disposed proximally of proximal base 16. Shaft assembly 12 further includes one or more proximal fixtures, e.g., proximal washer plate 74 and proximal lock plate 76 (FIGS. 3-7), disposed proximally of proximal articulating section 17 and disposed in fixed engagement within housing 20.

Distal and proximal articulating sections 15, 17 each include a respective plurality of articulation links 18a, 18b (FIG. 3). A plurality of articulation cables 19 are fixed at their proximal end portions to proximal lock plate 76 (FIGS. 3-7), and at their distal end portions to distal segment 13. Articulation cables 19 extend through shaft assembly 12, including each of the plurality of articulation links 18a, 18b (FIG. 3), such that articulation of housing 20 (and, thus, proximal lock plate 76 (FIGS. 3-7)) relative to proximal base 16 about articulation links 18b affects articulation of distal segment 13 (and, thus, end effector assembly 100) relative to proximal segment 14 about articulation links 18a between, for example, an un-articulated position (FIG. 1A), wherein housing 20, shaft assembly 12, and end effector assembly 100 are generally aligned on a longitudinal axis, and an articulated position (FIG. 1B), wherein housing 20, a portion of shaft assembly 12 (e.g., distal segment 13 and at least a portion of distal articulating section 15), and end effector assembly 100 are articulated off of the longitudinal axis. Articulation cables 19 may be routed in any suitable manner, such that an input articulation to articulation links 18b results in a desired output articulation of articulation links 18b, e.g., in the same direction, opposite directions, etc.

Handle assembly 30 of surgical instrument 10 includes a fixed handle 50 and a movable handle 40. Fixed handle 50 is integrally associated with housing 20 and handle 40 is movable relative to fixed handle 50. Movable handle 40 of handle assembly 30 is operably coupled to a drive assembly (not shown) that, together, mechanically cooperate to impart movement of one or both of jaw members 110, 120 of end effector assembly 100 about a pivot 103 between a spaced-apart position (FIG. 1A) and an approximated position (FIG. 1B) to grasp tissue between jaw members 110, 120. As shown in FIG. 1A, movable handle 40 is initially spaced-apart from fixed handle 50 and, correspondingly, jaw members 110, 120 of end effector assembly 100 are disposed in the spaced-apart position. Movable handle 40 is depressible from this initial position to a depressed position corresponding to the approximated position of jaw members 110, 120 (FIG. 1B).

Trigger assembly 60 includes a trigger 62 coupled to housing 20 and movable relative thereto between an un-actuated position and an actuated position. Trigger 62 is operably coupled to a cutting mechanism, e.g., a mechanical knife, an electrical cutter (static or movable), combinations thereof, etc., so as to actuate and/or activate the cutting mechanism to cut tissue grasped between jaw members 110, 120 of end effector assembly 100 upon actuation of trigger 62. As an alternative to a pivoting trigger 62, a slide trigger, push-button, toggle switch, or other suitable actuator may be provided.

End effector assembly 100, as noted above, includes first and second jaw members 110, 120. Each jaw member 110, 120 includes a proximal flange portion 111, 121, an outer insulative jaw housing 112, 122 disposed about the distal portion (not explicitly shown) of each jaw member 110, 120, and a tissue-treating plate 114, 124, respectively. Proximal flange portions 111, 121 are pivotably coupled to one another about pivot 103 for moving jaw members 110, 120 between the spaced-apart and approximated positions, although other suitable mechanisms for pivoting jaw members 110, 120 relative to one another are also contemplated. The distal portions (not explicitly shown) of the jaw members 110, 120 are configured to support jaw housings 112, 122, and tissue-treating plates 114, 124, respectively, thereon.

Outer insulative jaw housings 112, 122 of jaw members 110, 120 support and retain tissue-treating plates 114, 124 on respective jaw members 110, 120 in opposed relation relative to one another. Tissue-treating plates 114, 124 are formed from an electrically conductive material, e.g., for conducting electrical energy therebetween for treating tissue, although tissue-treating plates 114, 124 may alternatively be configured to conduct any suitable energy, e.g., thermal, microwave, light, ultrasonic, etc., through tissue grasped therebetween for energy-based tissue treatment. As mentioned above, tissue-treating plates 114, 124 are coupled to activation switch 4 and the source of energy (not shown), e.g., via the wires (not shown) extending from cable 2 through surgical instrument 10, such that energy may be selectively supplied to tissue-treating plate 114 and/or tissue-treating plate 124 and conducted therebetween and through tissue disposed between jaw members 110, 120 to treat tissue. One or both of jaw members 110, 120 may further define a longitudinally-extending channel 125 (only the channel of jaw member 120 is shown).

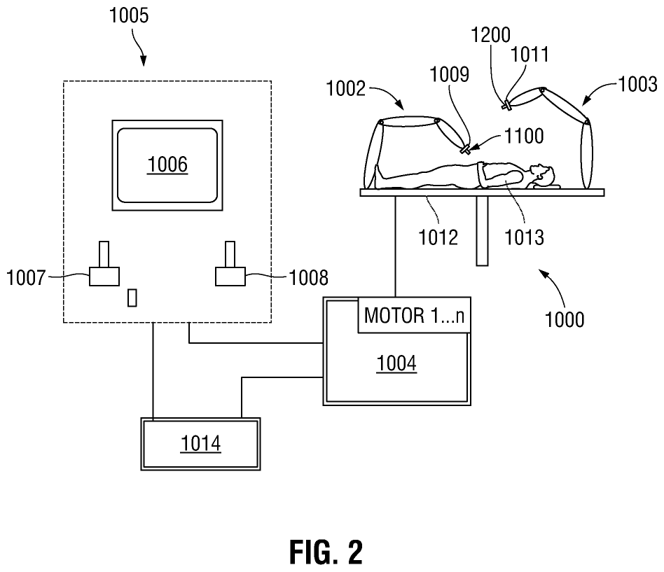

Referring generally to FIG. 2, a robotic surgical system exemplifying the aspects and features of the present disclosure is shown generally identified by reference numeral 1000. For the purposes herein, robotic surgical system 1000 is generally described. Aspects and features of robotic surgical system 1000 not germane to the understanding of the present disclosure are omitted to avoid obscuring the aspects and features of the present disclosure in unnecessary detail.

Robotic surgical system 1000 includes a plurality of robot arms 1002, 1003; a control device 1004; and an operating console 1005 coupled with control device 1004. Operating console 1005 may include a display device 1006, which may be set up in particular to display three-dimensional images; and manual input devices 1007, 1008, by means of which a surgeon may be able to telemanipulate robot arms 1002, 1003 in a first operating mode. Robotic surgical system 1000 may be configured for use on a patient 1013 lying on a patient table 1012 to be treated in a minimally invasive manner. Robotic surgical system 1000 may further include a database 1014, in particular coupled to control device 1004, in which are stored, for example, pre-operative data from patient 1013 and/or anatomical atlases.

Each of the robot arms 1002, 1003 may include a plurality of members, which are connected through joints, and an attaching device 1009, 1011, to which may be attached, for example, an end effector assembly 1100, 1200, respectively. End effector assembly 1100 may be similar to end effector assembly 100 (FIGS. 1A and 1B), although other suitable end effector assemblies for coupling to attaching device 1009 are also contemplated. Further, attaching device 1009 may include a shaft assembly similar to shaft assembly 12 (FIGS. 1A and 1B) that is operably coupled between robot arm 1002 and end effector assembly 1100 similarly as shaft assembly 12 is operably coupled between housing 20 and end effector assembly 100 (FIGS. 1A and 1B).

End effector assembly 1200 may be any end effector assembly, e.g., an endoscopic camera, graspers, scissors, or other surgical tool, etc. Robot arms 1002, 1003 and end effector assemblies 1100, 1200 may be driven by electric drives, e.g., motors, that are connected to control device 1004. Control device 1004 (e.g., a computer) may be configured to activate the motors, in particular by means of a computer program, in such a way that robot arms 1002, 1003, their attaching devices 1009, 1011, and end effector assemblies 1100, 1200 execute a desired movement and/or function according to a corresponding input from manual input devices 1007, 1008, respectively. Control device 1004 may also be configured in such a way that it regulates the movement of robot arms 1002, 1003 and/or of the motors.

Turning to FIGS. 3-7, shaft assembly 12 is described in greater detail. As noted above, shaft assembly 12 may interconnect housing 20 and end effector assembly 100 of surgical instrument 10 (FIGS. 1A and 1B). Shaft assembly 12 may similarly operate as attaching device 1009, interconnecting robot arm 1002 and end effector assembly 1100 of robotic surgical system 1000 (FIG. 2). Further, shaft assembly 12 may be configured to interconnect an end effector assembly and proximal support of any other suitable surgical instrument or system.

Continuing with reference to FIGS. 3-7, as noted above, shaft assembly 12 includes distal segment 13, proximal segment 14, distal articulating section 15 including articulation links 18a, proximal base 16, and proximal articulating section 17 including articulation links 18b. Shaft assembly further includes proximal washer plate 74 and proximal lock plate 76. Proximal segment 14, in aspects, may extend at least 30% of a length of shaft assembly 12, in other aspects, at least 35% of the length of shaft assembly 12, in other aspects, at least 40% of the length of shaft assembly 12. Distal segment 13, distal articulating section 15, proximal base 16, proximal articulating section 17, proximal washer plate 74, and proximal lock plate 76 define the remaining length of shaft assembly 12. In aspects, proximal and distal segments 14, 13 define substantially constant (wherein “substantially” accounts for measurement, manufacturing, material, environmental, etc. tolerances) outer diameters, which may be the same or different from one another. In aspects, distal articulating section 15 defines a substantially constant outer diameter (notwithstanding spaces between the articulation links 18a thereof), which may be the same or different from the outer diameters of proximal segment 14 and/or distal segment 13. The outer diameter of proximal segment 14 and/or distal segment 13 may, in embodiments, be less than about 4.0 mm (wherein “about accounts for measurement, manufacturing, material, environmental, etc. tolerances); in other embodiments, less than about 3.5 mm, in still other embodiments, less than about 3.3 mm; and, in yet other embodiments, less than about 3.1 mm. The outer diameter of distal articulating section 15 may be likewise defined. Thus, shaft assembly 12 may define an outer diameter of less than about 3.5 mm, less than about 3.3 mm, or less than about 3.1 mm over at least 30%, 35%, or 40% of the length thereof

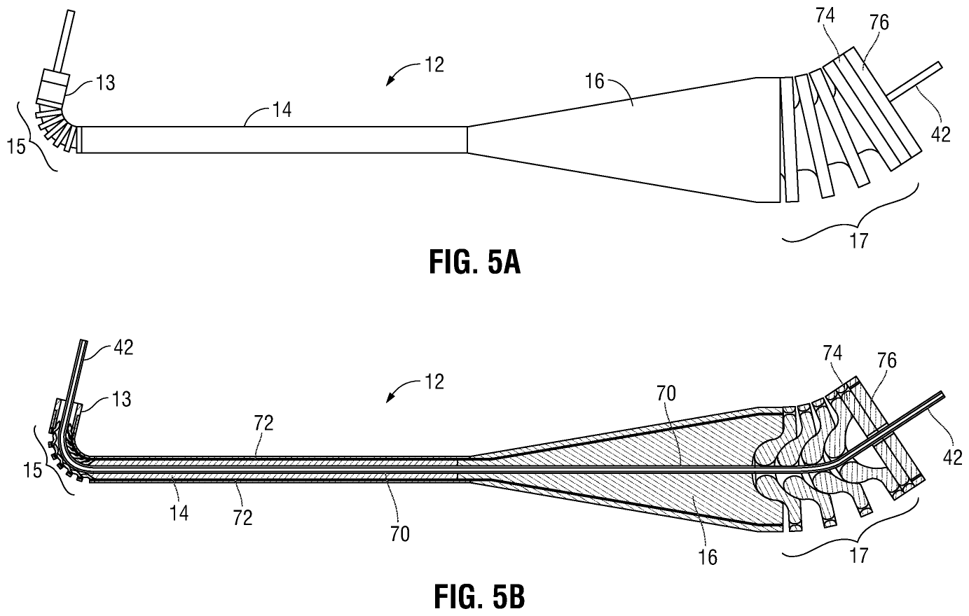

In aspects, proximal base 16 defines a varying outer diameter along at least a portion of its length. For example, the outer diameter of proximal base 16 may taper in a proximal-to-distal direction from a maximum outer diameter towards the proximal end of proximal base 16 to a minimum outer diameter towards the distal end of proximal base 16. The minimum outer diameter of proximal base 16, in aspects, may be substantially equal to the outer diameter of proximal segment 14 and/or distal segment 13. In embodiments, proximal articulating section 17 defines a substantially constant outer diameter (notwithstanding spaces between the articulation links 18b thereof), which may be substantially equal to the maximum outer diameter of proximal base 16. As a result of the configuration, proximal articulating section 17 includes articulation links 18b defining diameters greater than the diameters of articulation links 18a of distal articulating section 15 and, thus, amplification of articulation is achieved, e.g., wherein articulation of proximal articulating section 17 a first distance or angle results in articulation of distal articulating section 15 a second distance or angle greater than the first distance or angle (see FIGS. 5A and 5B). In embodiments, distal articulating section 15 may be configured to articulate to define a bend radius of at least about 4.5 mm; in other embodiments at least about 5.0 mm; and in still other embodiments at least about 5.5 mm. In embodiments, a bend ratio of distal articulating section 15, defined as the ratio of the bend radius to the outer diameter, is less than about 2.0. Proximal washer plate 74 and proximal lock plate 76 may define substantially equal outer diameters to proximal articulating section 17.

With reference to FIGS. 3, 4B, and 5B, distal segment 13, proximal segment 14, distal articulating section 15, proximal base 16, and proximal articulating section 17 cooperate to define a central longitudinal lumen 70 extending therethrough. With additional reference to FIGS. 1A and 1B, central longitudinal lumen 70 is configured to receive, for example, a drive sleeve 42 that is coupled between the drive assembly (not shown) and end effector assembly 100 (FIGS. 1A and 1B), and selectively translatable and/or rotatable within central longitudinal lumen 70 to impart movement of one or both of jaw members 110, 120 of end effector assembly 100 between a spaced-apart position (FIG. 1A) and an approximated position (FIG. 1B) to grasp tissue between jaw members 110, 120, e.g., upon actuation of movable handle 40. Central longitudinal lumen 70 may additionally or alternatively be configured to receive a knife drive rod 64 coupled between trigger assembly 60 and the cutting mechanism (not shown) and selectively translatable and/or rotatable within central longitudinal lumen 70 to impart movement of the cutting mechanism to cut tissue grasped between jaw members 110, 120 of end effector assembly 100, e.g., upon actuation of trigger 62. Additionally or alternatively, the wires (not shown) providing electrosurgical energy to tissue-treating plates 114, 124 may be routed through central longitudinal lumen 70.

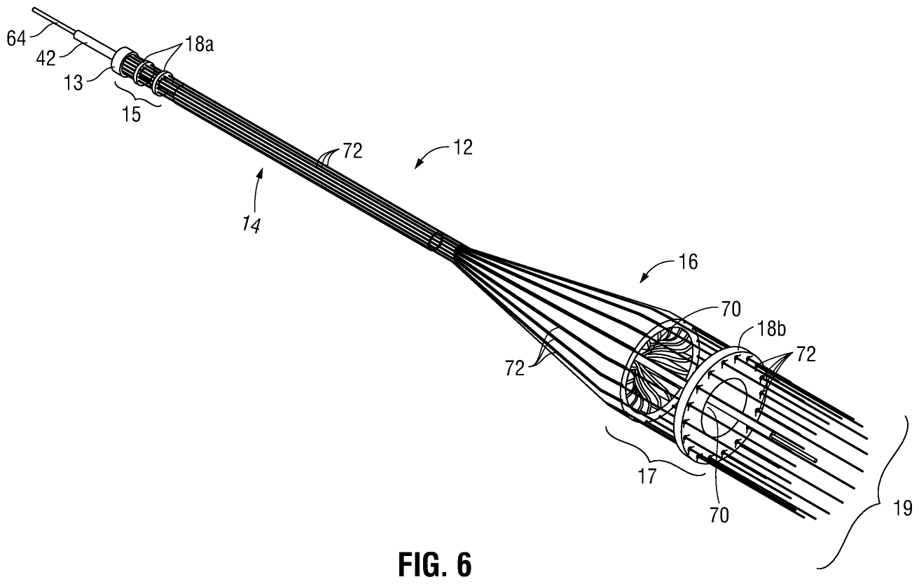

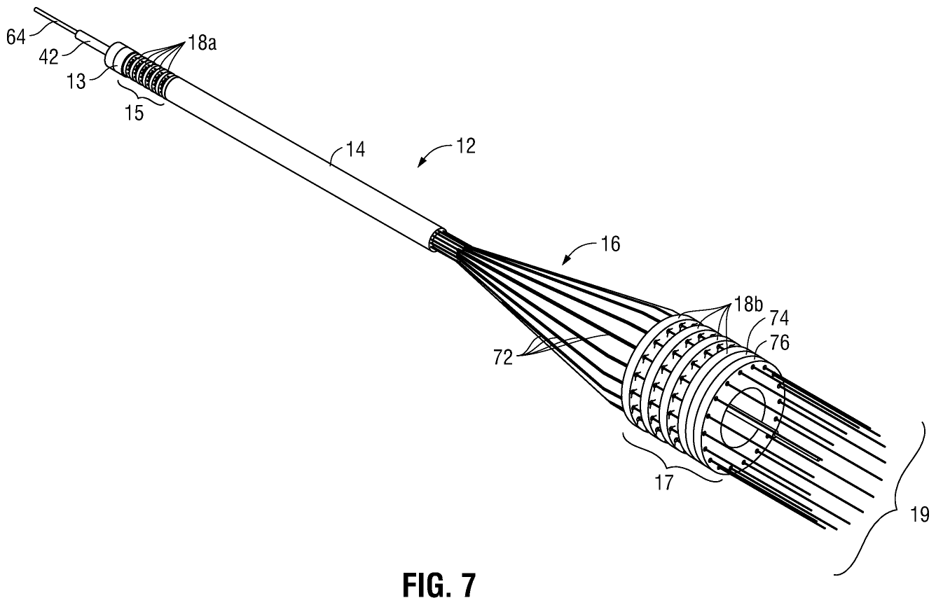

Proximal segment 14, distal articulating section 15, proximal base 16, and proximal articulating section 17 also cooperate to define a plurality of radial longitudinal lumens 72 extending therethrough and radially-spaced about central longitudinal lumen 70. The plurality of radial longitudinal lumens 72 extend into distal segment 13 and, in aspects, terminate therein, although the plurality of radial longitudinal lumens 72 may, in other aspects, also extend through distal segment 13. In embodiments, the plurality of radial longitudinal lumens 72 are formed as channels open to the annular peripheries of distal segment 13, proximal segment 14, distal articulating section 15, proximal base 16, and/or proximal articulating section 17 (see FIGS. 6 and 7). In other embodiments, only proximal base 16 includes longitudinal lumens 72 defining channels while the other components define fully enclosed longitudinal lumens 72. Further, in embodiments, radial longitudinal lumens 72 may extend in generally linear orientation relative to the longitudinal axis such that each lumen 72 starts and ends in substantially the same radial position or, in other embodiments, radial longitudinal lumens 72 may wind at least partially about the longitudinal axis, e.g., 180 degrees, such that each lumen 72 starts and ends at different, e.g., substantially diametrically opposed, radial positions.

In aspects, 18 radial longitudinal lumens 72 are provided, equally spaced radially about central longitudinal lumen 70 although other configurations are also contemplated, e.g., from 10 to 20 lumens 72, from 5 to 25 lumens, etc.

Each of distal segment 13, proximal segment 14, and proximal base 16 may be formed from a solid body (with the exception of lumens 70, 72 extending therethrough) and, in embodiments, may include an outer sleeve, e.g., shrink wrap, outer tube, or other suitable outer covering, disposed about the body. The solid bodies may be formed from a thermoplastic material or other suitable material and may be formed from injection molding or other suitable process. The outer sleeve may be continuous covering distal segment 13, proximal segment 14, proximal base 16, and articulating sections 15, 17, or may include separate portions covering one or more components and/or excluding one or more components.

The articulation links 18a forming distal articulating section 15 and the articulation links 18b forming proximal articulating section 17 each include distally-facing semi-spherical protrusions and proximally-facing semi-spherical recesses. Each semi-spherical protrusion is at least partially received within the distally-adjacent semi-spherical recess such that each pair of adjacent articulation links 18a of distal articulating section 15 and each pair of adjacent articulation links 18b of proximal articulating section 17 defines a universal articulation joint, e.g., enabling omnidirectional articulation. Proximally-facing semi-spherical recesses may be defined within the proximal ends of distal segment 13 and/or proximal base 16 to define universal articulation joints between the distal-most articulation links 18a, 18b and distal segment 13 and proximal base 16, respectively. Likewise, a distally-facing semi-spherical protrusion may be defined at the distal end of proximal segment 14 to define a universal articulation joints between the proximal-most articulation links 18a and proximal segment 14. Other suitable articulating joint configurations, e.g., hinges, pivots, camming structures, flexible portions, are also contemplated.

Referring to FIGS. 6 and 7, in conjunction with FIGS. 3-5B, each articulation cable 19 extends through one of the radial longitudinal lumens 72 and, as such, eighteen (18) articulation cables 19 may be provided or, in other aspects, e.g., from ten (10) to twenty (20) articulation cables 19 may be provided, from five (5) to twenty-five (25) articulation cables 19 may be provided, from three (3) to four (4) articulation cables may be provided, etc. Each articulation cable 19 may be made from stainless steel, nitinol, or other suitable material.

A distal end portion of each articulation cable 19 is secured within distal segment 13, e.g., within the portion of the corresponding radial longitudinal lumen 72 extending into distal segment 13, in any suitable manner such as, for example, crimping, bonding, brazing, soldering, welding, via a set screw or other mechanical engagement, or in any other suitable manner. Articulation cables 19 extend proximally from distal segment 13, within respective radial longitudinal lumens 72, through articulation links 18a of distal articulating section 15, proximal segment 14, proximal base 16, and articulation links 18b of proximal articulating section 17.

Proximal washer plate 74 and proximal lock plate 76 are disposed proximally of articulation links 18b of proximal articulating section 17 and define central apertures 78 communicating with central lumen 70 and radially-arranged apertures 75, 77, respectively, communicating with radial longitudinal lumens 72 and configured to receive proximal end portions of articulation cables 19 therethrough. The proximal end portions of articulation cables 19, after tensioning of articulation cables 19 to a suitable pre-tension, are secured within apertures 77 of proximal lock plate 76 in any suitable manner such as, for example, crimping, bonding, brazing, soldering, welding, via a set screw or other mechanical engagement, or in any other suitable manner, to maintain the pre-tension on articulation cables 19. In embodiments where heat is utilized and/or generated during this securement, proximal washer plate 74 may serve as a thermal barrier to inhibit thermal damage to other components, e.g., articulation links 18b. In other aspects, proximal washer plate 74 may be omitted.

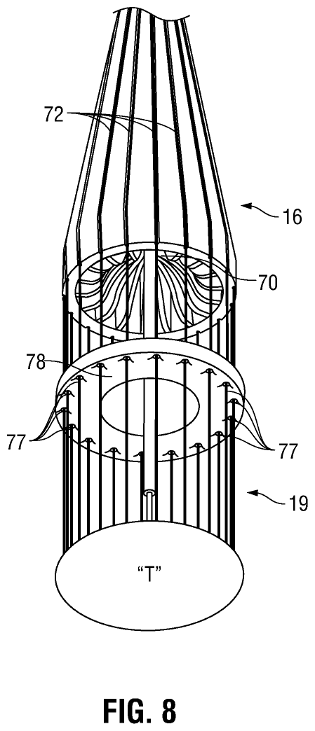

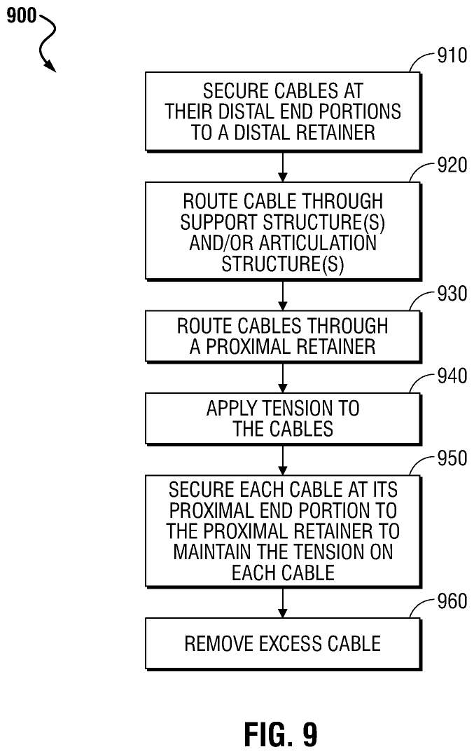

Turning now to FIGS. 8 and 9, a method, designated as 900, of assembling shaft assembly 12 while allowing independent and accurate pre-tension of each of articulation cables 19 is detailed. Initially, at 910, as noted above, the distal end portions of articulation cables 19 are secured within a distal retainer, e.g., respective radial longitudinal lumens 72 of distal segment 13 (see FIGS. 3 and 5). This may be accomplished via knotting the distal end portions of the articulation cables 19 to inhibit proximal passage through the distal retainer. Next at 920, prior to 910, or in simultaneous or overlapping temporal relation therewith, each articulation cables 19 is routed through support and/or articulation structures, e.g., radial longitudinal lumens 72 defined within distal segment 13, articulation links 18a of distal articulating section 15, proximal segment 14, proximal base 16, and articulation links 18b of proximal articulating section 17 (see FIGS. 3 and 5).

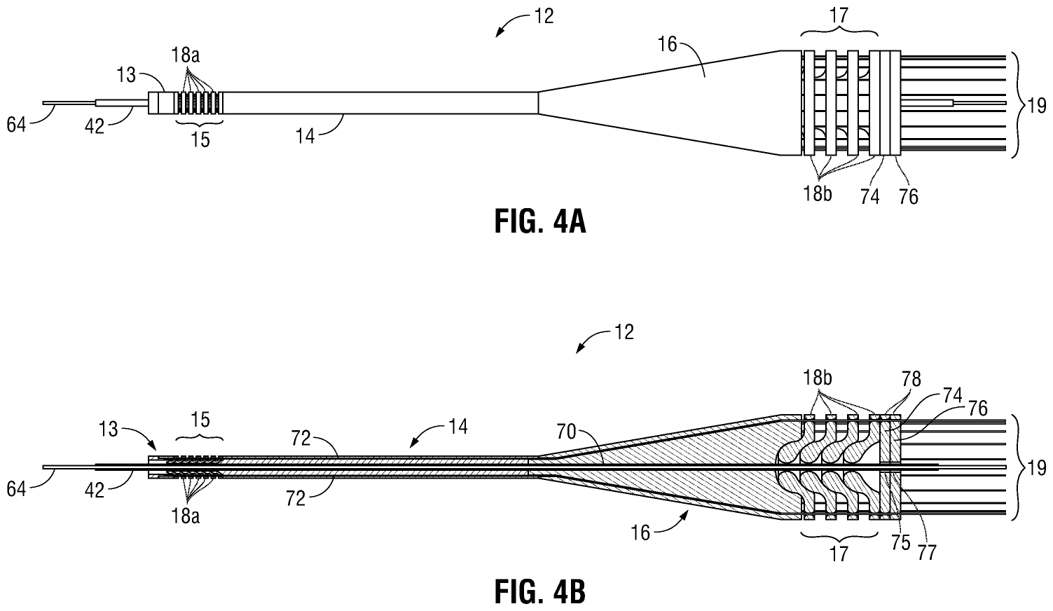

At 930, the proximal end portions of the articulation cables 19, once routed through the support and/or articulation structures, are routed through a proximal retainer, e.g., apertures 75, 77 of proximal washer plate 74 and proximal lock plate 76, respectively (see also FIGS. 3-4B), with the proximal end portions of the articulation cables 19 extending proximally from proximal lock plate 76. In some aspects, proximal washer plate 74 (FIGS. 3-4B) is omitted. Thereafter, at 940, tension is applied to each articulation cable 19, e.g., via one or more tensioners “T” attached to the proximal end portions of the articulation cables 19. The one or more tensioners “T” may include weights, tensioning fixtures, or other suitable tension-applying mechanism, and may include force gauges to indicate a tension on each of the articulation cables 19. Adjustment of the tension applied (individually to one or more of the articulation cables 19 or collectively to the plurality of articulation cables 19) may be performed to achieve a desired uniform pre-tension on each of the articulation cables 19.

Once the desired pre-tension on each of the articulation cables 19 is achieved at 940, the proximal end portion of each of the articulation cables 19 is secured to the proximal retainer, e.g., within the corresponding aperture 77 of proximal lock plate 76, at 950. This is accomplished individually for each articulation cable 19. This may be accomplished, for example, using set screws (not shown) to clamp the proximal end portions of the articulation cables 19 against interior surfaces (that define the corresponding apertures 77) of the proximal lock plate 76. Although each articulation cable 19 is individually secured, the securing may be performed individually for each cable 19, in groups of cables 19, or collectively for all cables 19. Other methods of attachment include crimping, bonding, brazing, soldering, welding, via another mechanical engagement, or in any other suitable manner. In this manner, the individual pre-tension on each cable 19 can be applied and maintained via the securement. Finally, at 960, once the articulation cables 19 are secured, excess portions of any cables 19 extending proximally from the proximal retainer, e.g., proximal lock plate 76, are cut-off and removed.

It is noted that while the above is described with respect to first securing the distal end portions of the articulation cables 19 to the distal retainer, routing the articulation cables 19, providing suitable tension on the articulation cables 19, and then securing the proximal end portions of the articulation cables 19 to the proximal retainer, it is also contemplated that the reverse may be accomplished. That is, the proximal end portions of the articulation cables 19 may first be secured to the proximal retainer, then routed, followed by pre-tensioning applied to the distal end portions of the articulation cables 19 before finally securing the distal end portions of the articulation cables 19 to the distal retainer.

It should be noted that the diameter difference between the tip and base may be configured to control overall articulation sensitivity. For example, if the tip and the base links are equal in location, they would have a 1:1 ratio. However, if the tip cables are in a smaller working diameter, there is a magnification of articulation from the input. Likewise, if the proximal end has cables in a smaller diameter, there is a reduction of motion at the distal tip.

It should be understood that various aspects disclosed herein may be combined in different combinations than the combinations specifically presented in the description and accompanying drawings. It should also be understood that, depending on the example, certain acts or events of any of the processes or methods described herein may be performed in a different sequence, may be added, merged, or left out altogether (e.g., all described acts or events may not be necessary to carry out the techniques). In addition, while certain aspects of this disclosure are described as being performed by a single module or unit for purposes of clarity, it should be understood that the techniques of this disclosure may be performed by a combination of units or modules associated with, for example, a medical device.

What is claimed is:

1. An articulating surgical instrument, comprising:
a distal retainer;
a proximal shaft segment;
a first articulating section extending between the distal retainer and the proximal shaft segment;
a proximal base extending proximally from the proximal shaft segment;
a proximal retainer;
a second articulating section extending between the proximal base and proximal retainer; and
a plurality of articulation cables secured at distal end portions thereof to the distal retainer and at proximal end portions thereof to the proximal retainer,
wherein the proximal shaft segment, the first articulating section, the proximal base, and the second articulating section cooperate to define a central longitudinal lumen therethrough and a plurality of radial longitudinal lumens therethrough that are radially disposed about the central longitudinal lumen, each articulation cable extending through one of the radial longitudinal lumens,
wherein articulation of the second articulating section affects articulation of the first articulating section.

2. The articulating surgical instrument according to claim 1, wherein the proximal base defines a solid body except for the portion of the central longitudinal lumen and portions of the radial longitudinal lumens extending therethrough.

3. The articulating surgical instrument according to claim 1, wherein the proximal shaft segment defines a solid except for the portion of the central longitudinal lumen and portions of the radial longitudinal lumens extending therethrough.

4. The articulating surgical instrument according to claim 1, wherein the proximal base tapers in diameter from a maximum diameter to a minimum diameter in a proximal-to-distal direction.

5. The articulating surgical instrument according to claim 4, wherein the first articulating section defines the minimum diameter and the second articulating section defines the maximum diameter such that articulation of the second articulating section affects amplified articulation of the first articulating section.

6. The articulating surgical instrument according to claim 1, wherein the radial longitudinal lumens are open channels along at least portions of lengths thereof.

7. The articulating surgical instrument according to claim 1, wherein the distal retainer, the proximal shaft segment, the first and second articulating sections, the proximal base, and the proximal retainer cooperate to define an overall length, and wherein a constant outer diameter of less than about 3.5 mm is defined along at least 30% of the overall length.

8. The articulating surgical instrument according to claim 1, wherein the first articulating section defines a bend radius of at least 4.5 mm.

9. The articulating surgical instrument according to claim 1, wherein the first articulating section defines a bend ratio of less than about 2.0.

10. The articulating surgical instrument according to claim 1, further comprising at least one actuation component extending through the central longitudinal lumen.

11. The articulating surgical instrument according to claim 1, further comprising at least two actuation components extending through the central longitudinal lumen.

12. The articulating surgical instrument according to claim 1, wherein the first articulating section includes a plurality of articulation links configured to articulate omnidirectionally relative to one another.

13. The articulating surgical instrument according to claim 12, wherein each articulation link of the plurality of articulation links defines a semi-spherical recess at one end and semi-spherical protrusion at the other end.

14. A method of assembling an articulating surgical instrument, comprising:
securing a plurality of articulating cables within a distal retainer;
routing the plurality of articulation cables through at least one shaft segment and at least one articulating section;
routing the plurality of articulation cables through a proximal retainer such that proximal end portions of the plurality of articulation cables extend through and proximally from the proximal retainer, wherein each articulation cable of the plurality of articulation cables is routed separately through the proximal retainer;
applying tension at the proximal end portions of the plurality of articulation cables to thereby uniformly tension each articulation cable of the plurality of articulation cables; and
securing the proximal end portion of each articulation cable to the proximal retainer to maintain the uniform tension on each articulation cable.

15. The method of assembly according to claim 14, further comprising, after securing the proximal end portion of each articulation cable, cutting off any excess cable at the proximal end portion of each cable.

16. The method of assembly according to claim 14, wherein routing the plurality of articulation cables through the at least one shaft segment and at least one articulating section includes routing each articulation cable through a separate lumen.

17. The method of assembly according to claim 14, further comprising routing the plurality of articulation cables through a proximal washer before the proximal retainer, wherein the proximal washer provides a thermal barrier to distal components during the securing of the proximal end portions of each articulation cable to the proximal retainer.

18. The method of assembly according to claim 14, wherein the securing of the proximal end portions of each articulation cable to the proximal retainer includes at least one of: crimping, bonding, brazing, soldering, welding, or set screw engagement.

19. The method of assembly according to claim 14, wherein applying tension includes individually tensioning each of the articulation cables.

20. The method of assembly according to claim 19, wherein individually tensioning each of the articulation cables includes individually adjusting a tension on at least one of the articulation cables.
